(19) United States
(12) Patent Application Publication
(10) Pub. No.: US 2008/0097251 A1
(43) Pub. Date: Apr. 24, 2008

(54) Method and apparatus for treating vascular obstructions

(76) Inventor: Eilaz Babaev, Minnetonka, MN (US)

Correspondence Address: Bacoustics, LLC, 5929 BAKER ROAD, SUITE 470, MINNETONKA, MN, 55345, US

(21) Appl. No.: 11/454,018

(22) Filed: Jun. 15, 2006

(51) Int. Cl. A61N 7/00; A61B 18/02

(52) U.S. Cl. 601/2; 606/21

(57) ABSTRACT

Method and device for treating vascular obstruction using ultrasonic energy in combination with cryogenic energy and/or an expandable member is disclosed. Ultrasound energy is delivered from a specially designed ultrasound transducer that is inserted in a blood vessel. Ultrasound energy can be delivered in conjunction with cryogenic energy. Ultrasound energy can also be delivered in conjunction with an expandable member such as expandable tubing, a hinged transducer, or a balloon. Ultrasound energy can also be delivered in conjunction with both cryogenic energy and an expandable member. The use of ultrasound energy in combination with cryogenic energy and/or an expandable member can treat a vascular obstruction.

### BACKGROUND OF THE INVENTION

1. Field of the Invention

The present invention relates to method and apparatus for treating vascular obstructions by using ultrasound energy in conjunction with cryogenic energy and/or an expandable member.

2. Description of the Related Art

Vascular lesions have been traditionally treated by using percutaneous transluminal angioplasty (PTA) procedures, or more commonly known as “balloon” angioplasty. This procedure involves inserting a catheter with an expanding balloon into a blood vessel and positioning the balloon over the stenotic lesion to be treated. The balloon is then inflated to treat the lesion by compressing the lesion or stretching the walls of the blood vessel. One drawback of this method is that it does not remove the lesion or plaque. Restenosis can occur where the blood vessel narrows once again, which would then require another treatment. This technique can be used to treat both the coronary artery and other blood vessels. One problem with this procedure is that it relies on putting pressure on and possibly stretching the walls of the blood vessel. This in turn can cause stress on the blood vessel.

Balloon angioplasty has advanced into a method that also uses a cryoplasty balloon. See, for example, U.S. Pat. No. 5,868,735 to LaFontaine, U.S. Pat. No. 6,290,696 also to LaFontaine, and U.S. Pat. No. 6,290,696 to Joye. This method first uses balloon angioplasty treatment to compress the lesion. After the angioplasty treatment, a cryoplasty balloon is inflated and filled with a cooling fluid. The cooling fluid then delivers cool thermal energy through the cryoplasty balloon to the treatment area. The use of cryogenic energy to cool the area after treatment helps prevent restenosis in the blood vessel. Similar to the balloon angioplasty method described above, this method also relies on putting pressure on and possibly stretching the walls of the blood vessel.

Another method used to remove vascular lesions and blockages is ultrasonic angioplasty. This procedure involves inserting an ultrasonic catheter so that the catheter tip is positioned against the vascular blockage or lesion. The ultrasonic catheter is connected to an ultrasonic energy source via a transmission member or guide wire. Ultrasonic energy is delivered from the source, along the transmission member or wire, and to the ultrasonic catheter. The ultrasonic energy vibrates the ultrasonic catheter tip. This vibration in the catheter tip ablates and removes the vascular blockage or lesion by mechanical impact and cavitation. Because the ultrasonic energy must travel over a long distance, resulting in an attenuation of the energy, a great amount of ultrasonic energy must be delivered from the ultrasonic source. This can result in the ultrasound transmission member or wire breaking or fracturing during use. Additionally, the ultrasonic energy must be delivered at small intervals, generally through pulsed delivery, because of the risk of tissue damage from the heat thermal energy that is delivered as a result of using ultrasonic energy.

U.S. Pat. No. 5,474,530 to Passafar et al. and U.S. Pat. No. 5,324,255 to Passafar et al. disclose a method that uses ultrasonic angioplasty with balloon angioplasty. The ultrasound energy is used only to create a passage way through which a balloon catheter can travel if the opening in the blood vessel is not wide enough for the balloon catheter. Passafar's uses of ultrasound energy is only to create a passage for the balloon, and therefore still faces the drawback of the pressure on a blood vessel from an inflated balloon.

Current methods used to treat vascular obstruction rely on putting pressure on a blood vessel or delivery heat thermal energy to the blood vessel. These methods can result in stress on a blood vessel or in tissue damage from heat energy. Therefore, there is a need for a method and device that utilizes the benefits of ultrasonic energy to remove vascular obstructions but that does not pose the risk of heat thermal damage to the blood vessel. There is an additional need for a method and device that can utilize ultrasonic energy in conjunction with a balloon angioplasty device so that less pressure is exerted on the blood vessel from an inflated balloon. Finally, there is a need for a method and device that can combine the benefits of balloon angioplasty, ultrasonic angioplasty, and cryoplasty.

### SUMMARY OF THE INVENTION

The present invention is directed towards method and apparatus for treating vascular obstructions by using ultrasonic energy in conjunction with cryogenic energy and/or an expandable member. Method and apparatus in accordance with the present invention may meet the above-mentioned needs and also provide additional advantages and improvements that will be recognized by those skilled in the art upon review of the present disclosure.

The present invention comprises a specially designed ultrasound transducer. The transducer is inserted into a blood vessel to treat vascular obstructions. Examples of a vascular obstruction include, but are not limited to, plaque, lesion, thrombus, clot, and blockage. Treatment of a vascular obstruction includes methods such as removal, ablation, dilation, or other similar methods or combinations of methods. The transducer delivers ultrasound energy to treat a vascular obstruction. The ultrasound energy can be delivered directly to remove a vascular obstruction through mechanical vibration. The ultrasound energy can also be delivered through the fluid in the blood vessel to remove a vascular obstruction through cavitation.

The present invention allows for ultrasound energy to be delivered in conjunction with cryogenic energy. The use of cryogenic energy, when used in conjunction with ultrasound energy, may have multiple benefits. First, the cryogenic energy may cool the area to be treated in order to help loosen the obstruction that is being treated, which then may help the ultrasonic energy more easily, efficiently, and precisely treat the vascular obstruction. Second, the cryogenic energy may be used to protect the blood vessel. Delivering ultrasound energy can result in the delivery of heat energy to the blood vessel. The use of cryogenic energy may provide a cooling effect to prevent damage to the blood vessel that could result from the heat energy. This cooling effect may also allow for continuous delivery of ultrasonic energy rather than pulsed delivery because there may be less concern with the generation of heat energy. Additionally, the cryogenic energy may increase the effectiveness of the delivery of ultrasound energy. Finally, similar to its use with a balloon angioplasty device, the cryogenic energy may help prevent restenosis on the treated area.

The present invention also permits ultrasound energy to be used in conjunction with an expandable member. The expandable member may have a similar effect in treating a vascular obstruction as a balloon angioplasty device. Ultrasound energy, when used in conjunction with an expandable member, may allow for a more effective compression of a vascular obstruction. The use of ultrasound energy requires less pressure to be exerted from the expandable member, thereby reducing the stress imposed on a blood vessel. Furthermore, the ultrasound energy may be able to treat a full vascular occlusion at the same time the expandable member and/or ultrasound energy treat a partial vascular occlusion. The expandable member may be in different formats including, but not limited to, a balloon at the end of a transducer, a balloon inside a transducer, expandable tubing connecting the transducer to the proximal end, or a hinged transducer. The hinged transducer may open outward so that it may be able to exert more pressure on and ensure better contact with the obstruction being treated. Additionally, a balloon may be positioned inside the hinged transducer so that the balloon may inflate when the hinged transducer opens or separates.

The present invention finally permits ultrasound energy to be used in conjunction with both cryogenic energy and an expandable member. This combination may utilize the beneficial aspects of each of these individual methods described above, and therefore it may be more effective because it combines the beneficial aspects of all these methods rather than using any of the methods either individually or in pairs. The expandable member may again include, but is not limited to, a balloon at the end of the transducer, expandable tubing connecting the transducer to the proximal end of the ultrasound device, or a hinged transducer.

The invention is related to method and apparatus to treat vascular obstructions by using ultrasonic energy in combination with cryogenic energy and/or an expandable member One aspect of this invention may be to provide a method and device for more effective treatment of vascular obstructions.

Another aspect of the invention may be to provide a method and device for more efficient treatment of vascular obstructions.

Another aspect of the invention may be to provide a method and device that poses less risk of damage to blood vessels during the treatment of vascular obstructions.

These and other aspects of the invention will become more apparent from the written descriptions and figures below.

### BRIEF DESCRIPTION OF THE DRAWINGS

The present Invention will be shown and described with reference to the drawings of preferred embodiments and clearly understood in details.

FIG. 1 is a perspective view ultrasound apparatus with an ultrasonic transducer and elongated

FIGS. 2a-2m are front cross-sectional views of variations of an elongated tube.

FIG. 3a-3c are perspective views of the ultrasound energy as it emanates from the ultrasound transducer and ultrasound tip.

FIGS. 4a-4d are open perspective views of variations of the ultrasound transducer with an elongated tube.

FIGS. 5a-5b are perspective views of variations of a hinged transducer.

FIGS. 6a-6e are cross-sectional schematic views of an ultrasound apparatus with an expandable member.

FIGS. 7a-7b are embodiments of an ultrasound apparatus that has an internal power source.

### DETAILED DESCRIPTION OF THE INVENTION

The present invention is a method and apparatus for treating vascular obstructions by using ultrasonic energy in conjunction with cryogenic energy or an expandable member, or any combination thereof. Preferred embodiments of the present invention in the context of an apparatus and methods are illustrated in the figures and described in detail below.

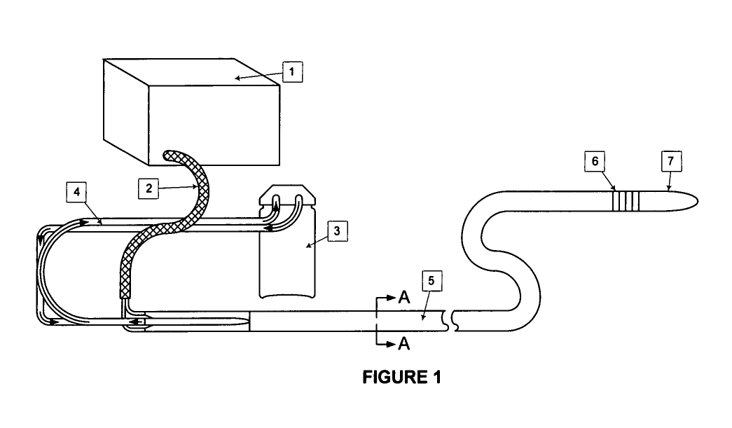

FIG. 1 is a perspective view of an ultrasound apparatus with an ultrasound transducer and an elongated tube/catheter for use according to the present invention. The apparatus is comprised of an ultrasound generator 1 that is connected to the transducer cable 2. This embodiment of the apparatus also comprises a cryogenic source 3 and a cryogenic tube 4. The transducer cable 2 and the cryogenic tube 4 are connected to the elongated tube 5. The elongated tube 5, which is connected to the ultrasound transducer 6, may serve as the delivery mechanism for the cryogenic energy from the cryogenic source 3 and for the electrical power from the ultrasound generator 1. The ultrasound transducer 6 is connected to the ultrasound tip 7. Other embodiments may be comprised of a fluid source instead of or in addition to the cryogenic source. A fluid such as saline or cryogenic energy may be used to enlarge an expandable member in the apparatus. Additionally, another embodiment could have neither a cryogenic source nor a fluid source.

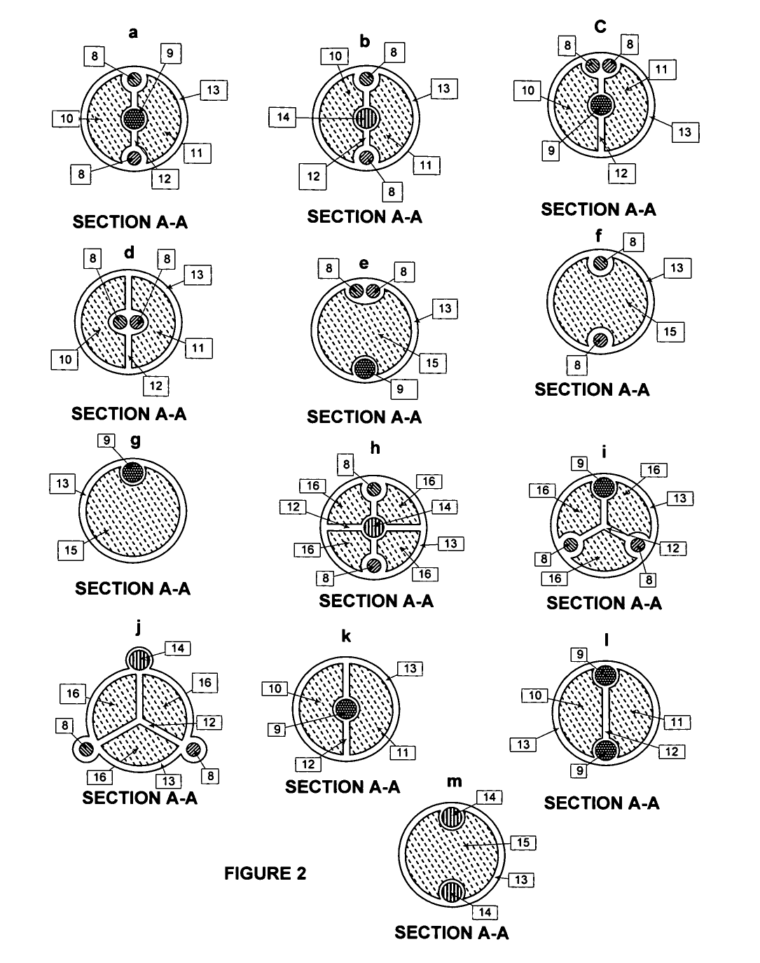

FIGS. 2a-2m are front cross-sectional views of variations of the elongated tube 5 for use according to the present invention.

FIG. 2a is an elongated tube 5 with electrical wires 8, a braided guide wire 9, a fluid entry lumen 10, a fluid exit lumen 11, inner tubing 12, and outer tubing 13. The electrical wires 8, positioned at the edges of the outer tubing 13 in this embodiment, act as the power source for the ultrasound transducer 6. The braided guide wire 9 is positioned in the center area of the elongated tube 5.

FIG. 2b is an elongated tube 5 with electrical wires 8, a fluid entry lumen 10, a fluid exit lumen 11, inner tubing 12, and outer tubing 13, and a solid guide wire 14. The braided guide wire 9 and the solid guide wire 14 may facilitate in the transmission of the elongated tube 5 through a blood vessel.

FIG. 2c is an elongated tube 5 with electrical wires 8, braided guide wire 9, a fluid entry lumen 10, a fluid exit lumen 11, inner tubing 12, and outer tubing 13. The electrical wires 8 in this embodiment are positioned along the same edge of the outer tubing 13.

FIG. 2d is an elongated tube with electrical wires 8, a fluid entry lumen 10, a fluid exit lumen 11, inner tubing 12, and outer tubing 13. The electrical wires 8 in this embodiment are located at the center area of the elongated tube 5 and act as a guide wire.

FIG. 2e is an elongated tube 5 with electrical wires 8, braided guide wire 9, outer tubing 13, and a single fluid lumen 15. The electrical wires 8 in this embodiment are located along the same edge of the outing tubing 13, and the braided guide wire 9 is located at another edge of the outer tubing 13. There is a single fluid lumen 15 that may allow for both the entry and the exit of a fluid.

FIG. 2f is an elongated tube 5 with electrical wires 8 and a single fluid lumen 15. In this embodiment, the electrical wires 8 are located at the edges of the outer tubing 13 and act as a guide wire.

FIG. 2g is an elongated tube with a braided guide wire 9 and a single fluid lumen 15. The braided guide wire 9 is located at the edges of the outer tubing 13. This embodiment does not contain electrical wires. An embodiment without electrical wires may be used with an ultrasound transducer that has an internal power source rather than an external power source with connecting electrical wires.

FIG. 2h is an elongated tube 5 with electrical wires 8 and a solid guide wire 14. In this embodiment, there are multiple fluid lumens 16. These fluid lumens 16 may be used in any number combination as fluid entry and fluid exit lumens. The fluid lumens 16 are divided by inner tubing 12. The solid guide wire 14 is located in the center area of the elongated tube, and the electrical wires 8 are located at the edges of the outer tubing 13.

FIG. 2i is an elongated tube 5 with electrical wires 8 and a braided guide wire 9. In this embodiment, there are multiple fluid lumens 16. These fluid lumens 16 may be used in any number combination as fluid entry and fluid exit lumens. The fluid lumens 16 are divided by inner tubing 12. The electrical wires 8 and braided guide wire 9 are located at the edges of the outer tubing 13.

FIG. 2j is an elongated tube 5 with electrical wires 8 and a solid guide wire 14. In this embodiment, there are multiple fluid lumens 16. The multiple fluid lumens 16 may be used in any number combination as fluid entry and fluid exit orifices. The fluid lumens 16 are divided by inner tubing 12. The solid guide wire 14 and the electrical wires 8 are located at the edges of the outer tubing 13.

FIG. 2k is an elongated tube 5, with a braided guide wire 9, a fluid entry lumen 10, a fluid exit lumen 11, inner tubing 12, and outer tubing 13. There are no electrical wires in this embodiment. The braided guide wire 9 is located in the center area of the elongated tube 5.

FIG. 2l is an elongated tube 5 with two braided guide wires 9 located along the edges of outer tubing 13, a fluid entry lumen 10, a fluid exit lumen 11, and inner tubing 12. There are no electrical wires in this embodiment.

FIG. 2m is an elongated tube 5 with two solid guide wires 14 located along the edges of outer tubing 13, and a single fluid lumen 15. There are no electrical wires in this embodiment.

FIGS. 2a-2m are only examples of variations of the elongated tube 5. Other similar embodiments or combinations of these embodiments may also be utilized. An elongated tube may comprise no guide wire, a single guide wire, or multiple guide wires. The tube may also comprise no electrical wires, a single electrical wire, or multiple electrical wires. The elongated tube may also comprise no lumen, a single lumen, or multiple lumens.

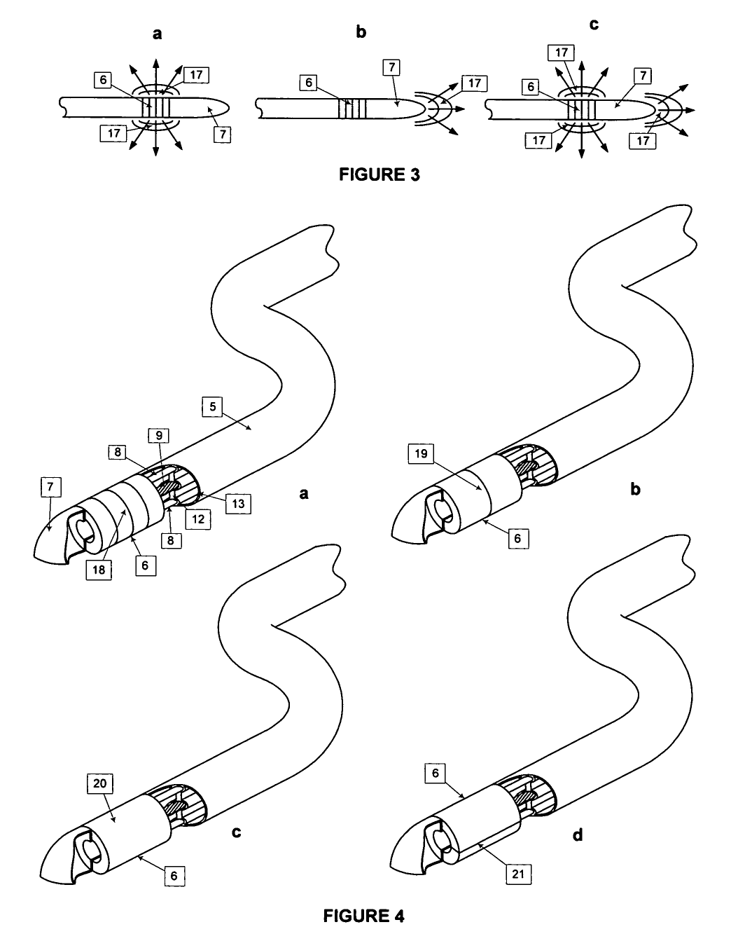

FIG. 3a-3c are perspective views of the ultrasound energy as it emanates from the ultrasound transducer 6 and ultrasound tip 7. FIG. 3a shows ultrasound energy 17 as it emanates from the radial side of the ultrasound apparatus. The ultrasound energy 17 that emanates from the radial side of an ultrasound transducer and/ultrasound tip are generally radial waves. FIG. 3b shows ultrasound energy 17 as it emanates from the distal end of the ultrasound apparatus. The ultrasound energy 17 that emanates from the distal end are generally longitudinal waves. FIG. 3c shows ultrasound energy 17 emanating from the ultrasound transducer 6 and ultrasound tip 7. Some shear waves may emanate along with the longitudinal and radial waves.

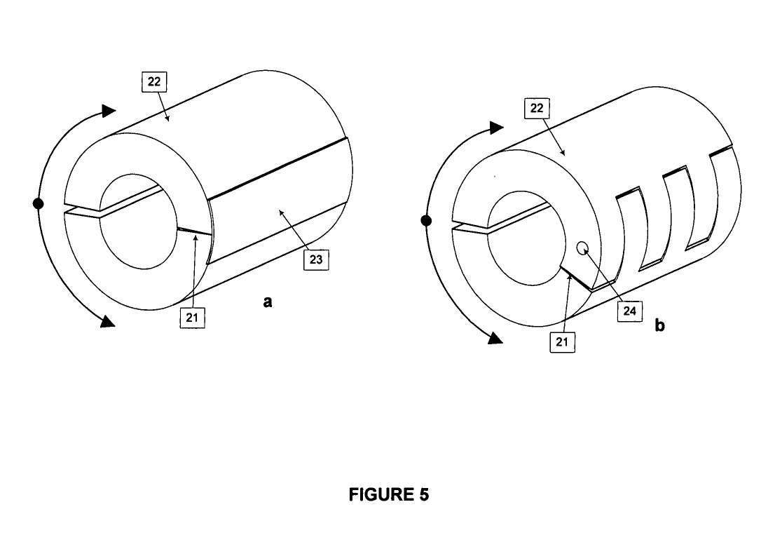

FIGS. 4a-4d are open perspective views of variations of the ultrasound transducer 6 for use according to the present invention. Each of the embodiments shown in these variations comprise an elongated tube 5, which is comprised of electrical wires 8, a braided guide wire 9, inner tubing 12, outer tubing 13, and an ultrasound tip 7. FIG. 4a is an open perspective view of an ultrasound apparatus comprised of an ultrasound transducer 6 that is comprised of multiple piezoelectric disks 18. FIG. 4b is an open perspective view of an ultrasound apparatus comprised of an ultrasound transducer 6 that is comprised of two piezoelectric disks 19. FIG. 4c is an open perspective view of an ultrasound apparatus comprised of an ultrasound transducer 6 that is comprised of one single piezoelectric disk 20. FIG. 4d is an open perspective view of an ultrasound apparatus comprised of an ultrasound transducer 6 that is comprised of two halves of piezoelectric disks 21. The two halves of piezoelectric disks 21 may be bonded together or hinged together for use as a hinged transducer, or the two halves of piezoelectric disks 21 may be separable.

FIGS. 5a and 5b are perspective views of variations of a hinged transducer 22. A hinged transducer 22 can be used as an expandable member according to the present invention. The hinged transducer 22 may open so that a balloon may expand from inside the transducer to contact the blood vessel. Additionally, the hinged transducer 22 may expand to contact the blood vessel itself without a balloon expanding. This direct contact may allow for more effective treatment of a vascular obstruction because of various benefits that may include opening the blood vessel, compressing an obstruction, and more effective delivery of ultrasound energy. FIG. 5a is a hinged ultrasound transducer 22, which is comprised of two separate halves of piezoelectric disks 21 that are connected via a thin membrane 23. FIG. 5b is a hinged ultrasound transducer 22, which is comprised of two separate halves of piezoelectric disks 21 that are connected via a pivot point 24.

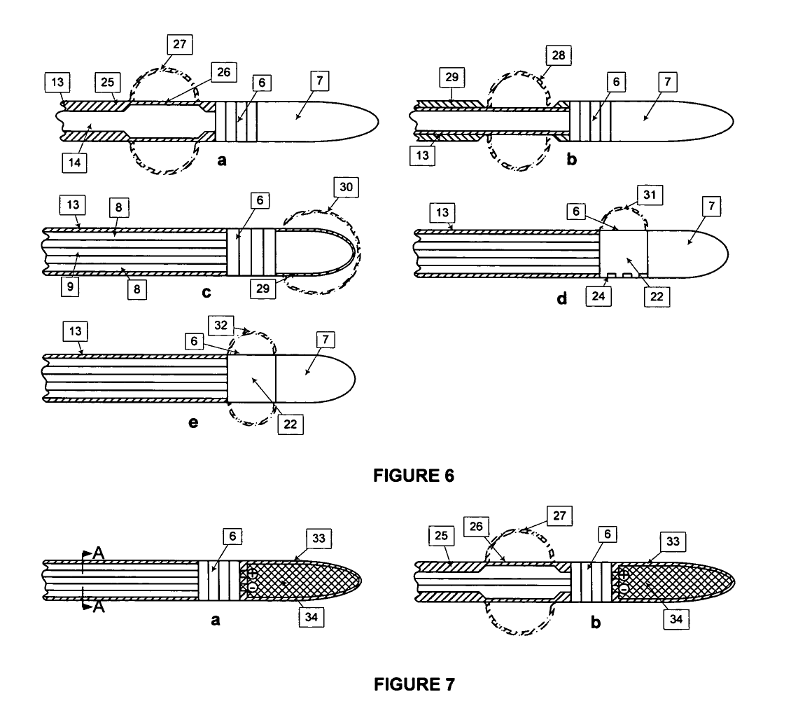

FIGS. 6a-6e are cross-sectional schematic views of an ultrasound apparatus with an expandable member. FIG. 6a is an ultrasound apparatus comprised of an ultrasound transducer 6 and an ultrasound tip 7. The elongated tube 5 is comprised of outer tubing 13, which is comprised of a thick section 25 and a narrow section 26. The thick section 25 is a certain thickness so that it remains a stable and is less expandable if a fluid flows through it. The narrow section 26 is thinner than the thick section 25 so that it is able to expand radially 27. In this embodiment, the narrow section 26 acts as an expandable member because it is able to expand radially 27. The elongated tube 5 in this embodiment is comprised of a single fluid lumen 14 as shown in FIG. 2e and FIG. 2f.

FIG. 6b is an ultrasound apparatus comprised of an ultrasound transducer 6 and an ultrasound tip 7. The outer tubing 13 is comprised so that it can expand 28 if a fluid flows through it. There is also a protective sheath 28 over the outer tubing 13 so that only a portion of outer tubing 13 is able to expand radially 27.

FIG. 6c is an ultrasound apparatus comprised of an ultrasound transducer 6 and an ultrasound tip 7. The outer tubing 13 is comprised of a certain thickness so that it remains a stable size if a fluid flows through it. This embodiment is comprised of an expandable member 29 is able to expand 30 at the distal end. The expandable member 29 could be expandable tubing, an inflatable balloon, or another similar expanding material. The elongated tube 5 in this embodiment is comprised of electrical wires 8 and a braided guide wire 9 as shown in FIG. 2a.

FIG. 6d is an ultrasound apparatus comprised of a hinged ultrasound transducer 22 and an ultrasound tip 6. The hinged transducer 22 is comprised of two halves of piezoelectric disks that are connected by a pivot point 24. The hinged ultrasound transducer 22 opens to allow an expandable member to expand 31. The expandable member with a hinged transducer 22 may be an inflatable balloon positioned inside the hinged transducer 22 that may be inflated. The expandable member may also be the hinged transducer 22 itself that opens to contact the walls of a blood vessel and/or a vascular obstruction.

FIG. 6e is an ultrasound apparatus comprised of an ultrasound transducer 6 and an ultrasound tip 7. In this embodiment, the ultrasound transducer 6 is comprised of two halves of piezoelectric disks 21 that are unconnected. The piezoelectric disks separate to allow an expandable member to expand 32. The expandable member may be an inflatable balloon located inside the transducer 6.

FIGS. 7a and 7b are embodiments of an ultrasound apparatus according to the present invention that are comprised of an internal power source. FIG. 7a depicts an ultrasound transducer 6 and an ultrasound tip 33 that has an internal power source 34. The internal power source 34 may be used in lieu of the external ultrasound generator 1 shown in FIG. 1. This embodiment does not comprise an expandable member. Cryogenic energy may still be delivered through the elongated tube 5 in conjunction with the ultrasound energy. FIG. 7b depicts an ultrasound transducer 6 and ultrasound tip 33 that has an internal power source 34. The elongated tube 5 is comprised of outer tubing 13, which is comprised of a thick section 25 and a narrow section 26. The thick section 25 is a certain thickness so that it remains a stable and is less expandable if a fluid flows through it. The narrow section 26 is thinner than the thick section 25 so that it is able to expand radially 27. In this embodiment, the narrow section 26 acts as an expandable member because it is able to expand radially 27.

The ultrasound apparatus shown in FIG. 1 delivers ultrasound energy to treat vascular obstructions. The present invention relates to a specially designed ultrasound transducer. The transducer is inserted into a blood vessel to treat vascular obstructions. Examples of vascular obstructions include, but are not limited to, plaques, lesions, thromboses, clots, and blockages. Treatment of a vascular obstruction includes methods such as removal, ablation, dilation, or other similar methods or combinations of methods. The transducer delivers ultrasound energy to treat a vascular obstruction. The ultrasound energy can be delivered directly or it can be delivered through the fluid in the blood vessel, thereby removing the vascular obstruction through mechanical vibration or cavitation. The ultrasound energy may be delivered from the radial side of the ultrasound transducer and/or ultrasound tip, from the distal end of the ultrasound tip, or from the enlargeable member, or any combination thereof. The ultrasound transducer may also be powered by an external power source such as an ultrasound generator or it my have an internal power source as shown in FIG. 7.

The present invention also relates to a specially designed elongated tube for use with the ultrasound transducer. The elongated tube is designed for inserting the ultrasound transducer into a blood vessel. The tube may also serve other functions that may include, but are not limited to, delivering the ultrasound power from the ultrasound generator to the transducer, delivering cryogenic energy, delivering fluid to enlarge an enlargeable member, or expanding radially to serve as an enlargeable member.

Ultrasound energy may be delivered in conjunction with cryogenic energy. The cryogenic energy may be delivered to the vascular obstruction and/or to the blood vessel; the cryogenic energy may be delivered through the elongated tube, the transducer, the ultrasound tip, or an expandable member. The cryogenic energy may be delivered before, during, and/or after the delivery of the ultrasonic energy.

The use of cryogenic energy, when used in conjunction with ultrasound energy, may have multiple benefits. First, the cryogenic energy may cool the area to be treated in order to help loosen the obstruction that is being treated, which then may help the ultrasonic energy more easily, efficiently, and precisely treat the vascular obstruction. Second, the cryogenic energy may be used to protect the blood vessel. Delivering ultrasound energy can result in the delivery of heat energy to the blood vessel. The use of cryogenic energy may provide a cooling effect to prevent damage to the blood vessel that could result from the heat energy. This cooling effect may also allow for continuous delivery of ultrasonic energy rather than pulsed delivery because there may be less concern with the generation of heat energy. Additionally, the cryogenic energy may increase the effectiveness of the delivery of ultrasound energy. Finally, similar to its use with a balloon angioplasty device, the cryogenic energy may help prevent restenosis on the treated area.

Ultrasound energy may be used in conjunction with an expandable member. The expandable member may have a similar effect in treating a vascular obstruction as a balloon angioplasty device. Ultrasound energy, when used in conjunction with an expandable member, may allow for a more effective compression of a vascular obstruction. The use of ultrasound energy requires less pressure to be exerted from the expandable member, thereby reducing the stress imposed on a blood vessel. Furthermore, the ultrasound energy may be able to treat a full vascular occlusion at the same time the expandable member and/or ultrasound energy treat a partial vascular occlusion. The expandable member may be in different formats including, but not limited to, a balloon at the end of a transducer, a balloon inside a transducer, expandable tubing connecting the transducer to the proximal end, or a hinged transducer. The hinged transducer may open outward so that it may be able to exert more pressure on and ensure better contact with the obstruction being treated. Additionally, a balloon may be positioned inside the hinged transducer so that the balloon may inflate when the hinged transducer opens or separates. Finally, ultrasonic energy may be used in conjunction with both cryogenic energy and an expandable member. This combination may utilize the beneficial aspects of each of these individual methods described above, and therefore it may be more effective because it combines the beneficial aspects of all these methods rather than using any of the methods either individually or in pairs. The expandable member may be comprised of a balloon at the end of the transducer, expandable tubing connecting the transducer to the proximal end, or a hinged transducer. Other expandable members may be similarly effective. The ultrasonic energy may be delivered before, during, or after enlarging the expandable member, or any combination thereof. The ultrasonic energy may also be delivered before, during, or after the delivery of cryogenic energy, or any combination thereof. The cryogenic energy may also be delivered before, during, or after enlarging of the expandable member, or any combination thereof.

The intensity of the ultrasound energy can be controlled through a variation in the ultrasound parameters such as the frequency, the amplitude, and the treatment time. The frequency range for the ultrasound energy is 16 kHz to 40 MHz. The low-frequency ultrasound range is 16 kHz-200 kHz, the preferred low-frequency ultrasound range is 30 kHz-100 kHz, and the recommend low-frequency ultrasound value is 80 kHz. The medium frequency ultrasound range is 200 kHz to 700 kHz, and the recommended medium frequency ultrasound value is 200 kHz. The high-frequency ultrasound range is 0.7 MHz-40 MHz, the more preferred high-frequency ultrasound range is 3 MHz-5 MHz, and the recommend high-frequency ultrasound value is 5 MHz. The amplitude of the ultrasound energy can be 1 micron and above. The preferred low-frequency ultrasound amplitude is in range of 2 microns to 250 microns, with the most preferred low-frequency amplitude to be in the range of 20 microns to 60 microns, and the recommended low-frequency amplitude value is 20-30 microns. The preferred amplitude range for of the high-frequency ultrasound is 1 micron to 10 microns, and the most preferred amplitude range for the high-frequency ultrasound is 2 microns to 5 microns. The preferred method of treatment uses low-frequency ultrasound.

Although specific embodiments and methods of use have been illustrated and described herein, it will be appreciated by those of ordinary skill in the art that any arrangement that is calculated to achieve the same purpose may be substituted for the specific embodiments and methods shown. It is to be understood that the above description is intended to be illustrative and not restrictive. Combinations of the above embodiments and other embodiments as well as combinations of the above methods of use and other methods of use will be apparent to those having skill in the art upon review of the present disclosure. The scope of the present invention should be determined with reference to the appended claims, along with the full scope of equivalents to which such claims are entitled.

### LITERATURE

### Articles

Hynynen, Kullervo et. al. Cylindrical Ultrasonic Transducers for Cardiac Catheter Ablation. IEEE Transactions on Biomedical Engineering. Vol. 44, No. 2: pg 144-51. February 1997.

U.S. Patent Documents

| | | |
|---|---|---|
| 3,433,226 | March, 1969 | Boyd |
| 3,565,062 | February, 1971 | Kuris |
| 3,823,717 | July, 1974 | Pohlman et al. |
| 4,808,153 | February, 1989 | Parisi |
| 4,841,977 | June, 1989 | Griffith et al. |
| 4,870,953 | October, 1989 | DonMicheal et al. |
| 4,924,863 | May, 1990 | Sterzer |
| 4,936,281 | June, 1990 | Stasz |
| 5,267,954 | December, 1993 | Nita |
| 5,304,115 | April, 1994 | Pflueger et al. |
| 5,324,255 | June, 1994 | Passafaro et al. |
| 5,326,342 | July, 1994 | Pflueger et al. |
| 5,334,181 | August, 1994 | Rubinsky et al. |
| 5,397,301 | March, 1995 | Pflueger et al. |
| 5,427,118 | June, 1995 | Nita et al. |
| 5,474,530 | December, 1995 | Passafaro et al. |
| 5,509,417 | April, 1996 | Dias et al. |
| 5,540,656 | July, 1996 | Pflueger et al. |
| 5,674,218 | October, 1997 | Rubinsky et al. |
| 5,713,916 | February, 1998 | Dias |
| 5,749,848 | May, 1998 | Jang et al. |
| 5,813,998 | September, 1998 | Dias |
| 5,836,896 | November, 1998 | Rosenschein |
| 5,868,735 | February, 1999 | Lafontaine |
| 5,899,899 | May, 1999 | Arless et al. |
| 5,934,284 | August, 1999 | Plaia et al. |
| 5,971,979 | October, 1999 | Joye et al. |
| 6,007,530 | December, 1999 | Dornhofer et al. |
| 6,012,457 | January, 2000 | Lesh |
| 6,024,740 | February, 2000 | Lesh et al. |
| 6,074,362 | June, 2000 | Jang et al. |
| 6,117,101 | September, 2000 | Diederich et al. |
| 6,149,596 | November, 2000 | Bancroft |
| 6,235,019 | May, 2001 | Lehmann et al. |
| 6,241,718 | June, 2001 | Arless et al. |
| 6,245,064 | June, 2001 | Lesh et al. |
| 6,254,599 | July, 2001 | Lesh et al. |
| 6,263,236 | July, 2001 | Kasinkas et al. |
| 6,270,493 | August, 2001 | Lalonde et al. |
| 6,283,959 | September, 2001 | Lalonde et al. |
| 6,290,696 | September, 2001 | Lafontaine |
| 6,355,029 | March, 2002 | Joye et al. |
| 6,383,151 | May, 2002 | Diederich, et al. |
| 6,391,026 | May, 2002 | Hung et al. |
| 6,416,511 | July, 2002 | Lesh et al. |
| 6,423,089 | July, 2002 | Gingras et al. |
| 6,428,534 | August, 2002 | Joye et al. |
| 6,432,102 | August, 2002 | Joye et al. |
| 6,450,975 | September, 2002 | Brennan et al. |
| 6,468,297 | October, 2002 | Williams et al. |
| 6,471,683 | October, 2002 | Drasler et al. |
| 6,478,754 | November, 2002 | Babaev |
| 6,482,218 | November, 2002 | Tran |
| 6,494,891 | December, 2002 | Cornish et al. |
| 6,508,781 | January, 2003 | Brennan et al. |
| 6,514,245 | February, 2003 | Williams et al. |
| 6,517,536 | February, 2003 | Hooven et al. |
| 6,524,251 | February, 2003 | Rabiner et al. |
| 6,527,767 | March, 2003 | Wang et al. |
| 6,533,803 | March, 2003 | Babaev |
| 6,537,271 | March, 2003 | Murray et al. |
| 6,540,740 | April, 2003 | Lehmann et al. |
| 6,546,935 | April, 2003 | Hooven |
| 6,558,366 | May, 2003 | Drasler et al. |
| 6,569,099 | May, 2003 | Babaev |
| 6,575,933 | June, 2003 | Wittenberger et al. |
| 6,575,966 | June, 2003 | Lane et al. |
| 6,589,253 | July, 2003 | Cornish et al. |
| 6,595,988 | July, 2003 | Wittenberger et al. |
| 6,595,989 | July, 2003 | Schaer |
| 6,601,581 | August, 2003 | Babaev |
| 6,602,246 | August, 2003 | Joye et al. |
| 6,602,247 | August, 2003 | Lalonde |
| 6,602,276 | August, 2003 | Dobak, III et al. |
| 6,623,444 | September, 2003 | Babaev |
| 6,629,972 | October, 2003 | Lehmann et al. |
| 6,645,202 | November, 2003 | Pless et al. |
| 6,648,878 | November, 2003 | Lafontaine |
| 6,648,879 | November, 2003 | Joye et al. |
| 6,652,547 | November, 2003 | Rabiner et al. |
| 6,660,013 | December, 2003 | Rabiner et al. |
| 6,663,554 | December, 2003 | Babaev |
| 6,663,622 | December, 2003 | Foley et al. |
| 6,666,858 | December, 2003 | Lafontaine |
| 6,669,689 | December, 2003 | Lehmann et al. |
| 6,669,693 | December, 2003 | Friedman |
| 6,673,066 | January, 2004 | Werneth |
| 6,673,068 | January, 2004 | Berube |
| 6,673,090 | January, 2004 | Root et al. |
| 6,685,702 | February, 2004 | Quijano et al. |
| 6,685,732 | February, 2004 | Kramer |
| 6,702,811 | March, 2004 | Stewart et al. |
| 6,712,816 | March, 2004 | Hung et al. |
| 6,723,064 | April, 2004 | Babaev |
| 6,752,805 | June, 2004 | Maguire et al. |
| 6,758,830 | July, 2004 | Schaer et al. |
| 6,761,729 | July, 2004 | Babaev |
| 6,780,183 | August, 2004 | Jimenez, Jr. et al. |
| 6,786,900 | September, 2004 | Joye et al. |
| 6,786,901 | September, 2004 | Joye et al. |
| 6,805,128 | October, 2004 | Pless et al. |
| 6,805,129 | October, 2004 | Pless et al. |
| 6,811,544 | November, 2004 | Schaer |
| 6,811,550 | November, 2004 | Holland et al. |
| 6,814,730 | November, 2004 | Li |
| 6,815,694 | November, 2004 | Sfez et al. |
| 6,849,075 | February, 2005 | Bertolero et al. |
| 6,875,209 | April, 2005 | Zvuloni et al. |
| 6,866,670 | March, 2005 | Rabiner et al. |
| 6,889,694 | May, 2005 | Hooven |
| 6,896,673 | May, 2005 | Hooven |
| 6,905,494 | June, 2005 | Yon et al. |
| 6,908,462 | June, 2005 | Joye et al. |
| 6,913,604 | July, 2005 | Mihalik et al. |
| 6,923,806 | August, 2005 | Hooven et al. |
| 6,932,811 | August, 2005 | Hooven et al. |
| 6,939,348 | September, 2005 | Malecki et al. |
| 6,942,659 | September, 2005 | Lehmann et al. |
| 6,955,174 | October, 2005 | Joye et al. |
| 6,972,015 | December, 2005 | Joye et al. |
| 6,974,454 | December, 2005 | Hooven |
| 6,984,233 | January, 2006 | Hooven |
| 6,989,009 | January, 2006 | Lafontaine |
| 7,001,378 | February, 2006 | Yon et al. |
| 7,001,383 | February, 2006 | Keidar |
| 7,001,415 | February, 2006 | Hooven |
| 7,008,411 | March, 2006 | Mandrusov et al. |
| 7,022,120 | April, 2006 | Lafontaine |

I claim:

1) A method for ultrasonic angioplasty with an expandable member, comprising the steps of:
a) inserting an ultrasound transducer with an expandable member into a blood vessel;
b) positioning the ultrasound transducer on and/or near a vascular obstruction;
c) enlarging the expandable member; and
d) delivering ultrasound to the vicinity of a vascular obstruction;
e) wherein the ultrasound is capable of treating a vascular obstruction.

2) The method according to claim 1, further comprising the step of generating said ultrasound.

3) The method according to claim 1, wherein said ultrasound comprises low-frequency ultrasound with a frequency within the approximate range of 16 kHz-200 kHz.

4) The method according to claim 1, wherein said ultrasound comprises low-frequency ultrasound with a preferred frequency within the approximate range of 30 kHz-100 kHz.

5) The method according to claim 1, wherein said ultrasound comprises low-frequency ultrasound with a recommended frequency of approximately 80 kHz.

6) The method according to claim 1, wherein said ultrasound comprises medium-frequency ultrasound with a frequency within the approximate range of 200 kHz-700 kHz.

7) The method according to claim 1, wherein said ultrasound comprises medium-frequency ultrasound with a recommended frequency of approximately 200 kHz.

8) The method according to claim 1, wherein said ultrasound comprises high-frequency ultrasound with a frequency within the approximate range of 700 kHz-40 MHz.

9) The method according to claim 1, wherein said ultrasound comprises high-frequency ultrasound with a preferred frequency within the approximate range of 3 MHz-5 MHz.

10) The method according to claim 1, wherein said ultrasound comprises high-frequency ultrasound with a recommended frequency of approximately 5 MHz.

11) The method according to claim 1, wherein the ultrasound amplitude is at least 1 micron.

12) The method according to claim 1, wherein said ultrasound comprises low-frequency ultrasound with an amplitude within the approximate range of 2 microns-250 microns.

13) The method according to claim 1, wherein said ultrasound comprises low-frequency ultrasound with a preferred amplitude within the approximate range of 20 microns-60 microns.

14) The method according to claim 1, wherein said ultrasound comprises low-frequency ultrasound with a recommended amplitude of approximately 20 microns-30 microns.

15) The method according to claim 1, wherein said ultrasound comprises medium-frequency ultrasound with a preferred amplitude within the approximate range of 2 microns-60 microns.

16) The method according to claim 1, wherein said ultrasound comprises medium-frequency ultrasound with a most preferred amplitude within the approximate range of 5 microns-30 microns.

17) The method according to claim 1, wherein said ultrasound comprises medium-frequency 15 ultrasound with a recommended amplitude of approximately 5 microns-10 microns.

18) The method according to claim 1, wherein said ultrasound comprises high-frequency ultrasound with a preferred amplitude within the approximate range of 1 micron-10 microns.

19) The method according to claim 1, wherein said ultrasound comprises high-frequency ultrasound with a most preferred amplitude within the approximate range of 2 microns-5 microns.

20) The method according to claim 1, wherein enlarging the expandable member is in the manner of radially expanding an elongated tube.

21) The method according to claim 1, wherein enlarging the expandable member is in the manner of expanding a hinged transducer.

22) The method according to claim 1, wherein enlarging the expandable member is in the manner of inflating a balloon.

23) The method according to claim 1, wherein the ultrasound is delivered before, during, or after enlarging the expandable member, or any combination thereof.

24) A method for ultrasonic cryoplasty, comprising the steps of:
a) Inserting an ultrasonic transducer into a blood vessel
b) Positioning the ultrasonic transducer on or near a vascular obstruction;
c) Delivering ultrasound to the vicinity of a vascular obstruction; and
d) Delivering cryogenic energy to the vicinity of a vascular obstructioon;
e) Wherein the ultrasound is capable of treating a vascular obstruction.

25) The method according to claim 24, further comprising the step of generating said ultrasound.

26) The method according to claim 24, further comprising the step of generating said cryogenic energy wherein said generated cryogenic energy is capable of enhancing the removal of a vascular obstruction.

27) The method according to claim 24, wherein said ultrasound comprises low-frequency ultrasound with a frequency within the approximate range of 16 kHz-200 kHz.

28) The method according to claim 24, wherein said ultrasound comprises low-frequency ultrasound with a preferred frequency within the approximate range of 30 kHz-100 kHz.

29) The method according to claim 24, wherein said ultrasound comprises low-frequency ultrasound with a recommended frequency of approximately 80 kHz.

30) The method according to claim 24, wherein said ultrasound comprises medium-frequency ultrasound with a frequency within the approximate range of 200 kHz-700 kHz.

31) The method according to claim 24, wherein said ultrasound comprises medium-frequency ultrasound with a recommended frequency of approximately 200 kHz.

32) The method according to claim 24, wherein said ultrasound comprises high-frequency ultrasound with a frequency within the approximate range of 700 kHz-40 MHz.

33) The method according to claim 24, wherein said ultrasound comprises high-frequency ultrasound with a preferred frequency within the approximate range of 3 MHz-5 MHz.

34) The method according to claim 24, wherein said ultrasound comprises high-frequency ultrasound with a recommended frequency of approximately 5 MHz.

35) The method according to claim 24, wherein the ultrasound amplitude is at least 1 micron.

36) The method according to claim 24, wherein said ultrasound comprises low-frequency ultrasound with an amplitude within the approximate range of 2 microns-250 microns.

37) The method according to claim 24, wherein said ultrasound comprises low-frequency ultrasound with a preferred amplitude within the approximate range of 20 microns-60 microns.

38) The method according to claim 24, wherein said ultrasound comprises low-frequency ultrasound with a recommended amplitude of approximately 20 microns-30 microns.

39) The method according to claim 24, wherein said ultrasound comprises medium-frequency ultrasound with a preferred amplitude within the approximate range of 2 microns-60 microns.

40) The method according to claim 24, wherein said ultrasound comprises medium-frequency ultrasound with a most preferred amplitude within the approximate range of 5 microns-30 microns.

41) The method according to claim 24, wherein said ultrasound comprises medium-frequency ultrasound with a recommended amplitude of approximately 5 microns-10 microns.

42) The method according to claim 24, wherein said ultrasound comprises high-frequency ultrasound with a preferred amplitude within the approximate range of 1 micron -10 microns.

43) The method according to claim 24, wherein said ultrasound comprises high-frequency ultrasound with a most preferred amplitude within the approximate range of 2 microns-5 microns.

44) The method according to claim 24, wherein the ultrasound is delivered before, during, or after the delivery of the cryogenic energy, or any combination thereof.

45) A method for ultrasonic cryoplasty with an expandable member, comprising the steps of:
a) Inserting an ultrasound transducer into a blood vessel;
b) Positioning the ultrasound transducer on or near a vascular obstruction;
c) Enlarging an expandable member;
d) Delivering ultrasound to the vicinity of a vascular obstruction; and
e) Delivering cryogenic energy to the vicinity of a vascular obstruction.
f) Wherein the ultrasound is capable of treating a vascular obstruction.

46) The method according to claim 45, further comprising the step of generating said ultrasound.

47) The method according to claim 45, further comprising the step of generating cryogenic energy, wherein said cryogenic energy is capable of enhancing the treatment of a vascular obstruction.

48) The method according to claim 45, wherein said ultrasound comprises low-frequency ultrasound with a frequency within the approximate range of 16 kHz-200 kHz.

49) The method according to claim 45, wherein said ultrasound comprises low-frequency ultrasound with a preferred frequency within the approximate range of 30 kHz-10 kHz.

50) The method according to claim 45, wherein said ultrasound comprises low-frequency ultrasound with a recommended frequency of approximately 80 kHz.

51) The method according to claim 45, wherein said ultrasound comprises medium-frequency ultrasound with a frequency within the approximate range of 200 kHz-700 kHz.

52) The method according to claim 45, wherein said ultrasound comprises medium-frequency ultrasound with a recommended frequency of approximately 200 kHz.

53) The method according to claim 45, wherein said ultrasound comprises high-frequency ultrasound with a frequency within the approximate range of 700 kHz-40 MHz.

54) The method according to claim 45, wherein said ultrasound comprises high-frequency ultrasound with a preferred frequency within the approximate range of 3 MHz-5 MHz.

55) The method according to claim 45, wherein said ultrasound comprises high-frequency ultrasound with a recommended frequency of approximately 5 MHz.

56) The method according to claim 45, wherein the ultrasound amplitude is at least 1 micron.

57) The method according to claim 45, wherein said ultrasound comprises low-frequency ultrasound with an amplitude within the approximate range of 2 microns-250 microns.

58) The method according to claim 45, wherein said ultrasound comprises low-frequency ultrasound with a preferred amplitude within the approximate range of 20 microns-60 microns.

59) The method according to claim 45, wherein said ultrasound comprises low-frequency ultrasound with a recommended amplitude of approximately 20 microns-30 microns.

60) The method according to claim 45, wherein said ultrasound comprises medium-frequency ultrasound with a preferred amplitude within the approximate range of 2 microns-60 microns.

61) The method according to claim 45, wherein said ultrasound comprises medium-frequency ultrasound with a most preferred amplitude within the approximate range of 5 microns-30 microns.

62) The method according to claim 45, wherein said ultrasound comprises medium-frequency ultrasound with a recommended amplitude of approximately 5 microns-10 microns.

63) The method according to claim 45, wherein said ultrasound comprises high-frequency ultrasound with a preferred amplitude within the approximate range of 1 micron -10 microns.

64) The method according to claim 45, wherein said ultrasound comprises high-frequency ultrasound with a most preferred amplitude within the approximate range of 2 microns-5 microns.

65) The method according to claim 45, wherein the ultrasound is delivered before, during, or after the delivery of the cryogenic energy, or any combination thereof.

66) The method according to claim 45, wherein the cryogenic energy is delivered before, during, or after enlarging of the expandable member, or any combination thereof.

67) The method according to claim 45, wherein the ultrasound is delivered before, during, or after enlarging of the expandable member, or any combination thereof.

68) The method according to claim 45, wherein enlarging the expandable member is in the manner of radially expanding an elongated tube.

69) The method according to claim 45, wherein enlarging the expandable member is in the manner of expanding a hinged transducer.

70) The method according to claim 45, wherein enlarging the expandable member is in the manner of inflating a balloon.

71) An ultrasound device for treating a vascular obstruction, comprised of
a) an ultrasound power source and a transducer for producing ultrasound energy;
b) wherein the ultrasound transducer is specially designed for insertion into a blood vessel;
c) wherein the ultrasound transducer delivers ultrasound energy to the vicinity of a vascular obstruction; and
d) wherein the ultrasound is capable of treating a vascular obstruction.

72) The apparatus according to claim 71, wherein the power source and transducer generate the ultrasound energy with particular ultrasound parameters indicative of an intensity capable of treating a vascular obstruction.

73) The method according to claim 71, wherein said ultrasound comprises low-frequency ultrasound with a frequency within the approximate range of 16 kHz-200 kHz.

74) The method according to claim 71, wherein said ultrasound comprises low-frequency ultrasound with a preferred frequency within the approximate range of 30 kHz-10 kHz.

75) The method according to claim 71, wherein said ultrasound comprises low-frequency ultrasound with a recommended frequency of approximately 80 kHz.

76) The method according to claim 71, wherein said ultrasound comprises medium-frequency ultrasound with a frequency within the approximate range of 200 kHz-700 kHz.

77) The method according to claim 71, wherein said ultrasound comprises medium-frequency ultrasound with a recommended frequency of approximately 200 kHz.

78) The method according to claim 71, wherein said ultrasound comprises high-frequency ultrasound with a frequency within the approximate range of 700 kHz-40 MHz.

79) The method according to claim 71, wherein said ultrasound comprises high-frequency ultrasound with a preferred frequency within the approximate range of 3 MHz-5 MHz.

80) The method according to claim 71, wherein said ultrasound comprises high-frequency ultrasound with a recommended frequency of approximately 5 MHz.

81) The method according to claim 71, wherein the ultrasound amplitude is at least 1 micron.

82) The method according to claim 71, wherein said ultrasound comprises low-frequency ultrasound with an amplitude within the approximate range of 2 microns-250 microns.

83) The method according to claim 71, wherein said ultrasound comprises low-frequency ultrasound with a preferred amplitude within the approximate range of 20 microns-60 microns.

84) The method according to claim 71, wherein said ultrasound comprises low-frequency ultrasound with a recommended amplitude of approximately 20 microns-30 microns.

85) The method according to claim 71, wherein said ultrasound comprises medium-frequency ultrasound with a preferred amplitude within the approximate range of 2 microns-60 microns.

86) The method according to claim 71, wherein said ultrasound comprises medium-frequency ultrasound with a most preferred amplitude within the approximate range of 5 microns-30 microns.

87) The method according to claim 71, wherein said ultrasound comprises medium-frequency ultrasound with a recommended amplitude of approximately 5 microns-10 microns.

88) The method according to claim 71, wherein said ultrasound comprises high-frequency ultrasound with a preferred amplitude within the approximate range of 1 micron -10 microns.

89) The method according to claim 71, wherein said ultrasound comprises high-frequency ultrasound with a most preferred amplitude within the approximate range of 2 microns-5 microns.

90) The ultrasound device according to claim 71, wherein the power source is internal in the transducer.

91) The ultrasound device according to claim 71, wherein the power source is external to the transducer.

92) The ultrasound device according to claim 71, further comprised of a fluid source.

93) The ultrasound device according to claim 92, wherein the fluid source is a cryogenic source.

94) The ultrasound device according to claim 71, further comprised of an elongated tube connecting the ultrasound transducer to the proximal end of the ultrasound device.

95) The ultrasound device according to claim 71, further comprised of an expandable member.

96) The ultrasound device according to claim 95, wherein the expandable member is a hinged transducer.

97) The ultrasound device according to claim 95, wherein the expandable member is an inflatable balloon.

98) The ultrasound device according to claim 95, wherein the balloon is positioned on the distal end of the transducer.

99) The ultrasound device according to claim 95, wherein the expandable member is a radially expandable elongated tube connecting the transducer to the proximal end of the ultrasound device.

100) An elongated tube comprised of:
a) Outer tubing;
b) An internal lumen or lumens;
c) An internal guide wire or guide wires;
d) wherein the internal lumen or lumens are capable of delivering a fluid; and
e) wherein the guide wire or guide wires are capable of facilitating the transmission of the elongated tube through a blood vessel.

101) The elongated tube according to claim 100, wherein the guide wire or guide wires are solid, braided, or another similarly effective form.

102) The elongated tube according to claim 100, further comprised of electrical wires.

103) The elongated tube according to claim 100, wherein the guide wire or guide wires are electrical wires.

104) The elongated tube according to claim 100, wherein the outer tubing is made of an expandable material, a non-expandable material, or a combination of expandable and non-expandable material.

105) The elongated tube according to claim 100, further comprised of inner tubing.

106) The elongated tube according to claim 100, further comprised of a sheath over the outer tubing.

107) The elongated tube according to claim 100, wherein the sheath covers a portion of the outer tube.
